(19) United States
(12) Patent Application Publication
(10) Pub. No.: US 2023/0030365 A1
(43) Pub. Date: Feb. 2, 2023

(54) BUOYANT PHOTOBIOREACTOR ARRANGEMENT

(71) Applicant: Technische Universitaet Hamburg, Hamburg (DE)

(72) Inventors: Leonard Francke, Hamburg (DE); Kerstin Kuchta, Hamburg (DE); Nils Wieczorek, Hamburg (DE); Sarah Loehn, Hamburg (DE)

(21) Appl. No.: 17/676,423

(22) Filed: Feb. 21, 2022

(30) Foreign Application Priority Data
Jul. 30, 2021 (DE) 20 2021 104 099 U

(51) Int. Cl. C12M 1/00; C12M 1/36; C12M 1/09; B63B 22/00; B63B 79/15

(52) U.S. Cl. CPC C12M 21/02; B63B 22/00; B63B 79/15; C12M 23/56; C12M 29/18; C12M 41/48

(57) ABSTRACT

The invention relates to a buoyant photobioreactor arrangement, wherein the photobioreactor arrangement is buoyant on a surface water, and comprises
a) a transparent photobioreactor container,
b) a floating body that is buoyant on the surface water and
c) a holding device arranged at or on the floating body holding the transparent photobioreactor container,
the photobioreactor container being able to be lowered into the surface water by means of the holding device.

### BACKGROUND OF THE INVENTION

### Field of the Invention

The invention relates to a buoyant photobioreactor arrangement.

### Description of the Related Art

As part of the increasing efforts to limit anthropogenic climate change by reducing greenhouse gas emissions and simultaneously to expand the supply of energy and raw materials in a sustainable and, above all, secure manner, the substitution of conventional, essentially fossil, resources and corresponding processes by new biotechnological processes and the use of renewable raw materials of various compositions and origins are becoming increasingly important. The mass cultivation of microalgae can play a decisive role here. Since said cultivation can be done in a self-sufficient area, microalgae have many advantages over conventional energy and industrial plants, which advantages can be developed through the use of special production systems, such as closed transparent photobioreactors (PBR) or open circulation systems (raceway ponds).

Microalgae themselves are tiny, photosynthetically active organisms having high growth rates that, by means of photosynthesis, convert solar energy and CO₂ into organic compounds such as essential fatty acids, valuable protein building blocks or pigments. These compounds can then be used as raw materials in a large number of products. In addition, microalgae are able to accumulate storage materials such as lipids or carbohydrates in high concentrations and are therefore considered to be a potential raw material for future biogenic energy carriers.

Despite these properties, commercial use has so far been limited to a few species and products, such as, for example, astaxanthin from Haematococcus pluvialis. In addition to the fact that many algae technologies are still in a relatively early stage of development due to insufficient knowledge of physiological processes and mechanisms of action, and difficulties due to complex approval procedures, this is also largely due to the lack of economic attractiveness due to the current costs of cultivation and processing methods.

Since microalgae need nutrients in order to grow, which nutrients arise as waste products in industrial processes and municipal wastewater, it is attractive to develop unused urban space as a cultivation area and thereby close material cycles. Coastal regions and port cities offer attractive locations, since unused surface water can be used as cultivation area. The waste streams from local industry and municipal wastewater can be used as nutrient streams for the microalgae. The biomass obtained can be used as raw material in a regional bioeconomy, thus closing the value chain.

So far, the cultivation and thus the production of microalgae has required a high level of energy. Photobioreactors, through the use of solar energy, heat up far beyond the tolerance limit of microalgae. This makes it necessary to cool the systems, for example, by means of shading or active cooling with water. The former entails severe losses in productivity, the latter is very energy-intensive.

It is therefore an object of the present invention to enable cultivation of microalgae which does not have the disadvantages of the prior art and in particular combines high productivity of the algae culture with a low energy consumption.

### BRIEF SUMMARY OF THE INVENTION

To achieve the object, the invention provides a photobioreactor arrangement, the photobioreactor arrangement being buoyant on surface water, comprising
a) a transparent photobioreactor container,
b) a floating body that can float on the surface water and
c) a holding device arranged at or on the floating body and holding the transparent photobioreactor container,
the photobioreactor container being able to be lowered into the surface water by means of the holding device.

The photobioreactor arrangement according to the invention enables efficient cultivation of microalgae by cultivating the microalgae in a photobioreactor container, which, on the one hand, can be exposed to intense natural solar radiation and high air temperatures, but on the other hand, can be efficiently cooled with the aid of a passive cooling system, so that high energy consumption through active cooling can be avoided. In the photobioreactor arrangement according to the invention, which is also referred to here as a “photobioreactor facility” or “photobioreactor system”, efficient passive cooling is made possible by lowering the photobioreactor container into surface water on which the photobioreactor arrangement having the photobioreactor container can be arranged floating. Sunlight reaches the microalgae culture in the transparent photobioreactor container, which sunlight can be used by the microalgae to form biomass by means of photosynthesis, preferably using inorganic carbon sources, such as CO₂. To cool the photobioreactor container and the microalgae culture therein, it is sufficient to lower the photobioreactor container sufficiently into the surface water and to allow the water of the surface water to wash around a sufficient area of the photobioreactor container. The lowering can take place to such an extent that sufficient cooling is made possible without losing too much sunlight through a layer of water located above the photobioreactor container. The temperature can be measured in the microalgae culture or the culture medium, for example, to determine whether there is sufficient cooling. This can optionally be combined, for example, with a measurement of the amount of light incident on or in the photobioreactor container.

A “photobioreactor container”, here optionally also simply referred to as “photobioreactor” (abbreviated to PBR) or “PBR container”, is understood to mean a bioreactor container which is used for the cultivation of phototrophic, preferably photolithoautotrophic, microorganisms, for example, algae, using light as an energy source. A “photobioreactor container” usually comprises a container wall that is permeable to light, at least light of the wavelength(s) required for photosynthesis, which container wall surrounds an inner cavity for receiving a culture medium for cultivating the microorganisms. The photobioreactor container can comprise means for circulating the culture, for example, a stirrer or pumps, and means for introducing and/or removing gases such as CO₂, for example, or acids or alkalis to adjust the pH.

The term “closed” in relation to the photobioreactor container means here that the photobioreactor container is closed off from the water of the surface water, so that the water cannot penetrate into the interior of the photobioreactor container. This does not rule out that the photobioreactor container has openings, connections or the like, via which, for example, a fluid, for example, a gas, for example, CO₂, or a liquid, can be introduced into or removed from the container.

The expression “buoyant on a surface water” means that the photobioreactor arrangement, when it is arranged on a surface water, does not sink, but rather drifts on the surface of the water. For this purpose, the photobioreactor arrangement is designed, for example, by means of suitable floating bodies, that its weight is less than the buoyancy force acting thereon.

A “floating body” is understood to mean any body whose weight is less than the buoyancy force acting on it in a fluid, for example, water. A floating body, for example, has a lower average density than the fluid, for example, water, or comprises a hollow body.

The expression “can be lowered into the surface water” in relation to the photobioreactor container means that the photobioreactor container can be at least partially, preferably, completely immersed in the body of water of the surface water, so that the photobioreactor container has at least part of its surface, preferably at least a part of its surface that is sufficient for sufficient cooling, for example, at least 10%, 20%, 30%, 40%, 50%, 60%, 70% or at least 80% of its surface, coming into contact with the body of water of the surface water. The term “can be lowered” includes the mobility of the photobioreactor container in a substantially vertical direction, that is, in the direction of gravity and against the direction of gravity, and is therefore not to be understood as meaning that the photobioreactor container can only be moved in the direction of the surface water and into the surface water, but such that the photobioreactor container can also be moved in the opposite direction and raised in relation to the surface water. “Can be lowered” includes that the photobioreactor container can be brought partially or completely below the water level of the surface water.

“Surface water” is understood to mean an above-ground or open body of water, with the exception of groundwater, which forms a water table on the earth’s surface. The term includes inland waters, transitional waters, coastal waters and seas or parts of seas. It can be fresh water, salt water or brackish water. The term includes both flowing waters and still waters. Examples of surface water are rivers, streams, lakes, ponds, etc.

The term “transparent” in relation to the “photobioreactor container” means that the wall of the photobioreactor container at least partially, preferably completely or at least predominantly, that is, at least 50%, 60%, 70%, 80% or at least 90%, consists of a material that is permeable at least to photosynthetically active radiation, that is, for light whose wavelength(s) is (are) necessary or suitable for photosynthesis by phototrophic microorganisms, in particular photosynthesis by microalgae, for example, light having wavelengths between 380 and 780 nm, 400 nm and 740 nm or 400 nm and 700 nm.

“Microalgae” are microscopic algae, that is, phototrophic eukaryotic microorganisms that are individually invisible to the naked eye. They can be single-cell or few-cell algae. Examples of microalgae are Tetradesmus obliquus, Chaetoceros sp., Chlorella vulgaris, Dunaliella salina and Haematococcus sp.

Surface waters are well suited for cooling a photobioreactor container, since their bodies of water usually have a water temperature that is sufficiently low for cooling, even in hot summer months. In addition, evaporation also causes sufficient cooling of the photobioreactor container.

In a preferred embodiment of the photobioreactor arrangement according to the invention
i) the floating body is arranged like a frame around the photobioreactor container and has a recess that accommodates the photobioreactor container,
ii) the holding device holding the photobioreactor container is arranged around the recess, and
iii) the photobioreactor container is held in the recess by the holding device such that it can be moved in the vertical direction.

In this embodiment, the floating body surrounds the photobioreactor container like a frame and encloses it horizontally, a recess being formed within the floating body, within which recess the photobioreactor container is arranged. The floating body can enclose the photobioreactor container such that a rectangular, square or also circular or elliptical recess is formed. It is preferred that the floating body surrounds the photobioreactor container completely, that is, over the entire circumference. However, it is also possible for the floating body to only partially surround the photobioreactor container, for example, 50%, 60%, 70%, 75%, 80%, 85%, 90% or 95%, so that there is still free access to the surface water in the horizontal direction. The floating body not only ensures that the photobioreactor arrangement floats on the surface water, but preferably also provides mechanical protection for the photobioreactor container. The photobioreactor container is therefore preferably enclosed by the floating body at least to such an extent that, for example, floating debris is held back in the surface water and damage to the photobioreactor container from the outside is prevented.

In a particularly preferred embodiment of the photobioreactor arrangement according to the invention, the photobioreactor container is tubular in form. In this embodiment, the photobioreactor container comprises a horizontally arranged, closed pipe or pipe system made of a preferably rigid, transparent wall material. For example, it can be a pipe or pipe system made of a transparent plastic or glass, for example, borosilicate glass. The material is preferably a plastic, for example, a vinyl polymer (polyvinyl chloride, PVC, for example, rigid PVC, PVC-U), polyacrylate (polymethyl methacrylate, PMMA), polycarbonate (PC) or polyethylene (PE). “Rigid” here means that it is essentially a stiff, inelastic material. However, it is also possible to use a plastic material that is flexible per se, for example, silicone, the wall thickness of which is designed such that the tubular system cannot collapse under normal circumstances. A “pipe system” is understood to mean a system composed of a plurality of individual pipes or pipe sections, the pipes or pipe sections being in fluid communication with one another. “Horizontal” here means that the longitudinal axes of the pipe or the pipes or pipe sections of the pipe system run essentially in a plane parallel to the surface of the surface water. The pipe or the pipes or pipe sections of the pipe system preferably have a round cross-section, but can also have a polygonal, rectangular, circular or elliptical cross-section. In this embodiment, the pipe or pipe system is closed and thus separated from the body of water of the surface water, so that no water from the surface water gets into the interior of the photobioreactor container. The horizontally arranged pipe or pipe system of the photobioreactor container can be moved in the vertical direction by means of the holding device and brought into contact with the water of the surface water in order to effect cooling of the photobioreactor container. For this purpose, the pipe or pipe system can only be lowered into the surface water to such an extent that sufficient cooling takes place. For this purpose, the pipe or pipe system can only be partially or completely lowered below the waterline of the surface water. The pipe or pipe system is preferably only lowered into the surface water to such an extent that, given sufficient cooling, there is still sufficient light irradiation for photosynthesis.

In this embodiment, the pipe or pipe system of the photobioreactor container is preferably arranged horizontally in a meandering shape. “Meandering-shaped” means that the pipe or pipe system has a curved or convoluted course.

The pipe or pipe system of the photobioreactor container is preferably connected to at least one pump unit, whereby the medium having the microalgae can be circulated. Particularly preferably, a collecting container is arranged between the pump unit and the photobioreactor container, which collecting container can also be used, for example, for harvesting the microalgae culture. The pump unit and the collecting container are preferably arranged in a stationary manner on the floating body. The pump unit can be connected to the photobioreactor container, for example, via hoses or the like, in order to create a closed system in which the microalgae culture can be circulated. The pump unit can comprise one or more pumps.

The photobioreactor arrangement according to the invention can comprise one or more, that is, one, two, three, four or more photobioreactor containers. In one embodiment, the photobioreactor arrangement according to the invention comprises a plurality of photobioreactor containers, that is, two or more photobioreactor containers, each of the photobioreactor containers being held by an individual holding device and each of the photobioreactor containers being able to be individually lowered into the surface water by means of the respective individual holding device. It is also possible to interconnect a plurality of photobioreactor arrangements according to the invention in a modular manner to form a larger system and to operate them in series or in parallel with one another.

In an alternative embodiment of a photobioreactor arrangement according to the invention having a plurality of photobioreactor containers, at least two of the photobioreactor containers are held by a common holding device and can be lowered together into the surface water by means of the common holding device.

The holding device holding the photobioreactor container is arranged at or on the floating body such that the photobioreactor container can be lowered into the surface water in a suitable manner. For this purpose, in one embodiment of the photobioreactor arrangement according to the invention having a recess in the floating body, it is advantageous if the holding device comprises a holding frame, for example, a metal frame, which is arranged around the recess. The holding frame can, for example, be arranged along the reveal of the recess lining the recess and fastened to or on the floating body in a suitable manner. The holding frame can therefore, for example, have the shape of the cross-section of the recess in cross-section and be designed, for example, rectangular, square, circular, elliptical or polyhedral. In principle, however, the holding frame does not have to follow the shape of the recess. The invention also encompasses embodiments in which the cross-sectional shape of the holding frame does not correspond to or follow the shape of the recess. The holding frame can consist of metal, for example, stainless steel, or other suitable materials, for example, plastic materials, or a combination thereof. The material is preferably as corrosion-resistant as possible. The photobioreactor container can be arranged on the holding frame so that it can be lowered and raised in any suitable manner. For example, the photobioreactor container can be arranged on a support frame dimensioned such that it can be moved within the holding frame perpendicular to the plane of the holding frame. In an embodiment of the photobioreactor arrangement according to the invention having a recess within the floating body, the support frame is preferably dimensioned such that it can be moved vertically in the recess. The support frame can comprise transverse and/or diagonal struts, for example, for more stable storage of the photobioreactor container. The support frame can, for example, be a metal frame, for example, an aluminum frame, which is equipped with sacrificial anodes, for example, magnesium sacrificial anodes, for cathodic protection against corrosion. The holding device preferably comprises a lifting system to which the photobioreactor container is fastened directly or indirectly and can be moved vertically within the holding frame. The lifting system can, for example, connect the holding frame and the support frame, so that the support frame with the photobioreactor container arranged thereon can be lowered or raised. The lifting system can comprise, for example, lifting columns that can be actuated manually or by a motor, for example, an electric motor, in order to lower or raise the photobioreactor container, which can, for example, be arranged on a support frame. Automatic control of the lift of the lifting columns is also possible.

In a preferred embodiment of the photobioreactor arrangement according to the invention, the photobioreactor container can be lowered manually into the surface water by means of the holding device. For this purpose, the lifting system can be equipped with manually actuated lifting columns, for example. In another preferred embodiment of the photobioreactor arrangement according to the invention, the photobioreactor container can also, as already mentioned above, be lowered or raised mechanically, for example, by means of motors, for example, electric motors.

The floating body of the photobioreactor arrangement according to the invention can be formed in one or more parts. In a preferred embodiment, the floating body is formed of a plurality of parts and comprises individual floating body modules, for example, in the form of a parallelepiped, which can be assembled to any shape, for example, a generally rectangular or square shape in cross-section, preferably having a recess. The floating body, in particular in an embodiment where a floating body surrounds a recess, can have a water passage in order to establish a connection between the water surface inside the recess and the water surface outside the recess. For this purpose, the floating body can have recesses on its underside, that is, regions in which the floating body does not rest on the water surface or in which it is not immersed in the surface water. This facilitates the exchange of water between the surface water inside the recess and outside the recess in order, for example, to avoid superficial heating of the part of the surface water within the recess. The floating body can also be designed in two or more parts, the two or more parts of the floating body not being connected to one another directly, but rather only being connected to one another via the holding device. For example, the floating body can be designed in two parts, the photobioreactor container being held between the two floating bodies so that it can be moved vertically with the aid of the holding device. The separate floating body parts can in turn be composed of a plurality of floating body modules. The floating body modules can be air-filled hollow plastic bodies, for example.

In a further preferred embodiment of the photobioreactor arrangement according to the invention, the photobioreactor arrangement comprises a measurement and control unit arranged on the floating body. The measurement and control unit comprises sensors, for example, temperature, flow, photo, pH, pO₂, pCO₂ sensors, which, for example, record important physical parameters of the culture medium or the environment, for example, temperature, pH value, oxygen and/or carbon dioxide concentration in the medium, temperature of the surface water, ambient air temperature, wind speed or light irradiation on or in the photobioreactor reactor. Photosensors can comprise, for example, pyranometers and sensors for photosynthetically active radiation (PAR sensors). The measurement and control unit can be used to control the cultivation conditions on the basis of the measured parameters, for example, by regulating the pH, pO₂ or pCO₂ value. Measurement data recorded by the measurement and control unit can also be sent wirelessly, for example, via radio (for example, WLAN), to a control system arranged outside the photobioreactor arrangement, which control system comprises a control program, for example, to provide remote control and/or to enable reading of measured values. The photobioreactor container preferably has, for example, a connection via which a carbon dioxide-air mixture, for example, a CO₂-air mixture having 10% (v/v) CO₂ content, can be introduced into the photobioreactor container, to improve the supply of CO₂ that is important for photosynthetic biomass production to the microalgae. In addition, the introduction of CO₂ into the photobioreactor container can also be used to regulate the pH value in order to preferably keep said value in the neutral range (pH 6-8). Optionally, the CO₂ content can also be adapted to the respective type of microalgae. The CO₂ can be provided from a pressurized gas cylinder or, for example, from an exhaust gas, for example, from a block-type thermal power station. Mixing with air can take place by means of a compressor. The gas can be supplied by means of pneumatic hoses.

In a particularly preferred embodiment of the photobioreactor arrangement according to the invention, said arrangement is designed and set up such that the photobioreactor container, based on measurement data supplied by sensors and captured by the measurement and control unit, for example, the temperature of the culture medium in the photobioreactor container, can be automatically lowered into the surface water. For example, the photobioreactor arrangement can be designed and set up so that when a maximum temperature of the culture medium is exceeded, the photobioreactor container is automatically lowered into the surface water, optionally taking into account the temperature of the surface water or the incidence of photosynthetically active radiation on the photobioreactor container. For this purpose, the measurement and control unit can be electrically connected to the lifting system of the holding device such that the lifting system can be controlled via the measurement and control unit. For this purpose, the lifting system can have motors, for example, electric motors, which are actuated automatically, for example, in the event that the desired maximum temperature is exceeded. The control can also take place such that the immersion depth of the photobioreactor container depends, for example, on the temperature of the culture medium and is dynamically adapted to the temperature. A control is also possible such that the immersion depth or, optionally, the immersion frequency of the photobioreactor container, is controlled using a plurality of parameters, for example, based on the temperature of the culture medium, the amount of photosynthetically active radiation incident on or in the photobioreactor container and the temperature of the surface water.

As already mentioned above, the photobioreactor arrangement according to the invention particularly preferably comprises a pump unit for circulating the medium in the photobioreactor container. The pump unit is preferably electrically connected to the measurement and control unit such that it can be controlled automatically via the measurement and control unit. For example, the photobioreactor arrangement can be set up such that the rate of circulation of the culture medium can be controlled by means of the measurement and control unit. The pump unit can also serve to better distribute a CO₂-air mixture introduced into the photobioreactor container in the medium. The gas mixture is introduced into the photobioreactor, for example, via pneumatic hoses, and the gas bubbles move through the photobioreactor container due to the circulating movement generated by the pump unit, the CO₂ dissolving in the culture medium and being able to be absorbed from the medium by the microalgae as a carbon source.

In a further preferred embodiment of the photobioreactor arrangement according to the invention, the photobioreactor container has an input and an output which are connected or can be connected by means of flexible lines, for example, hoses, to a collecting container arranged on the floating body and a pump unit preferably also arranged on the floating body. The input and output can each be openings having suitable connections for connecting the flexible lines, which can be, for example, pneumatic hoses, suction and pressure hoses and/or spiral hoses. In the case of a photobioreactor container designed as a pipe or pipe system, the input and output are preferably openings at the ends of the pipe or pipe system. The pump unit is preferably connected or connectable to the input and output of the photobioreactor container and the collecting container such that a culture medium accommodated in the photobioreactor container can be circulated via the collecting container by means of the pump unit. The pump unit and the collecting container are preferably connected to one another in fluid communication via lines, for example, plastic pipes. A flexible line is understood here as a line, for example, a hose or the like, which moves with a movement of the photobioreactor container, for example, a lowering or raising of the photobioreactor container, so that said flexible line remains connected to the photobioreactor container and does not tear off.

In order to provide the electrical energy required, for example, for the measurement and control unit and the pump unit, the photobioreactor arrangement according to the invention can comprise, for example, one or more solar collectors or one or more wind turbines. Solar collectors can, for example, be arranged on the surface of at least part of the floating body. An arrangement outside of the floating body is of course also possible, wherein the electrical connection to the measurement and control unit and the pump unit can be made by means of suitable electrical lines.

The photobioreactor arrangement according to the invention can advantageously be used in a method for cultivating a microalgae culture. The method preferably comprises the cultivation of a microalgae culture in a culture medium in a transparent photobioreactor container under irradiation with sunlight, the photobioreactor container being at least partially lowered into the body of water of a surface water for passive cooling of the photobioreactor container heated by solar irradiation.

The temperature of the microalgae culture or of the culture medium is preferably measured by means of sensors and the photobioreactor container is lowered into the body of water of the surface water as a function of the temperature of the microalgae culture, preferably automatically controlled. This temperature-controlled lowering of the photobioreactor container into the surface water ensures sufficient cooling. The lowering takes place only to the extent that sufficient cooling is enabled while there is still sufficient light irradiation. The light irradiation can, for example, also be measured by means of a photosensor, for example, a lux meter, pyranometer or a PAR sensor, and included in a regulation system, by means of which an optimal lowering position of the photobioreactor container can be determined. One, two or more further parameters, for example, the water temperature of the surface water, the ambient temperature or the wind speed can also be included in the control. The method can be designed, for example, so that the position of the photobioreactor container is kept, by means of a suitable control or regulation, in a range in which the amount of usable, that is, photosynthetically active radiation incident into the photobioreactor and the temperature of the culture medium for cultivation of the respective microalgae culture are optimal.

As already described above in more detail with regard to the photobioreactor arrangement according to the invention, the photobioreactor container can be lowered by means of a holding device which is arranged at or on a floating body that can float on the surface water and holds the photobioreactor container.

In a preferred embodiment of the method according to the invention, the photobioreactor container can also be cooled via evaporation. For this purpose, the photobioreactor container can be alternately lowered into the body of water of the surface water and then raised again.

### BRIEF DESCRIPTION OF THE SEVERAL VIEWS OF THE DRAWINGS

The invention is explained in more detail below, purely for illustrative purposes, with reference to the attached figures.

FIG. 1. Three-dimensional view of an embodiment of a photobioreactor arrangement according to the invention.

FIG. 2. Top view of the embodiment of a photobioreactor arrangement according to the invention depicted in FIG. 1.

FIG. 3. Three-dimensional view of part of the embodiment of a photobioreactor arrangement according to the invention depicted in FIG. 1.

FIGS. 4, 5. Detailed view of part of the lifting system of the embodiment of a photobioreactor arrangement according to the invention depicted in FIG. 1.

### DETAILED DESCRIPTION OF THE INVENTION

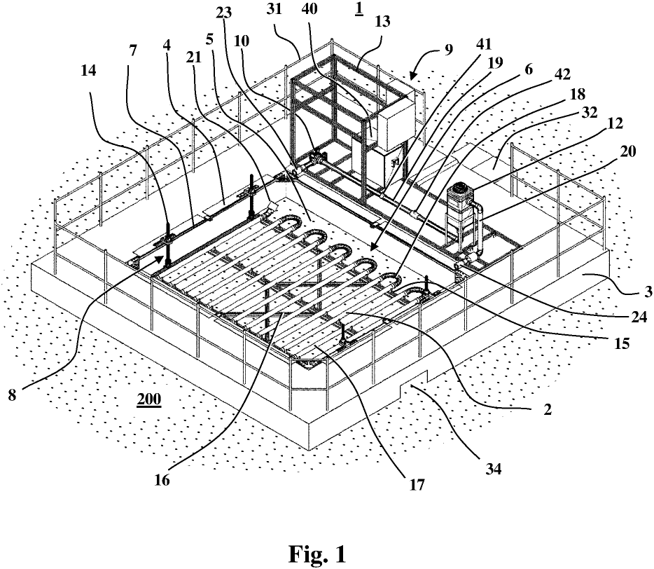

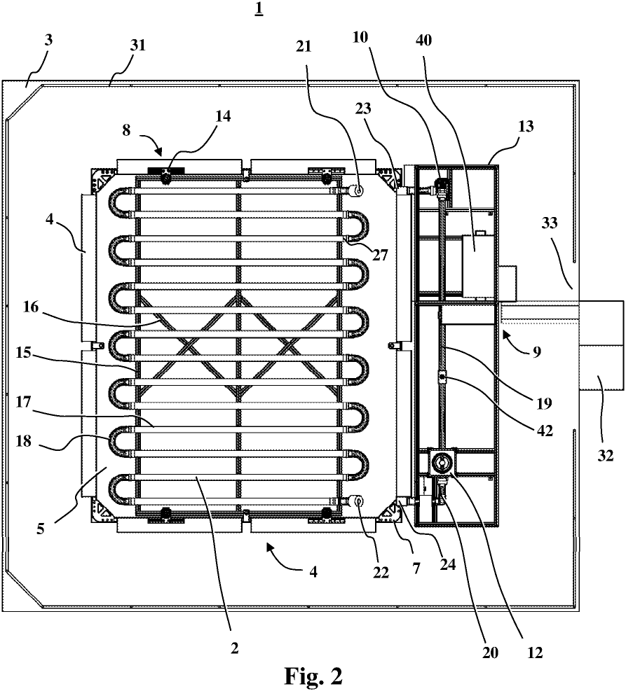

FIG. 1 shows a three-dimensional view of an embodiment of a photobioreactor arrangement 1 according to the invention floating on a surface water 200. The photobioreactor arrangement 1 here comprises a floating body 3 essentially rectangular in cross-section (see also FIG. 2), which floating body 3 is composed of individual cuboid floating body modules 32, of which only two are depicted here for the sake of clarity, which form a type of jetty on the outside. The floating body 3 has a recess 5. A photobioreactor container 2 is arranged inside the recess 5, which photobioreactor container 2 here consists of a meandering transparent pipe or pipe system 6 of straight cylindrical pipe elements 17 and curve elements 18 connected to one another with the aid of coupling elements 27. The recess 5 here lies almost centrally within the floating body 3, so that the floating body 3 is arranged in a frame-like manner around the photobioreactor container 2. A holding device 4 fastened around the recess 5 on the floating body 3 holds the transparent photobioreactor container 2 in the recess 5. The holding device 4 here comprises a metal holding frame 7 and a likewise metal support frame 15 on which the photobioreactor container 2 is mounted. The support frame 15 is stabilized by means of transverse/diagonal struts 16. Furthermore, the holding device 4 comprises a lifting system 8 having lifting columns 14, which here comprise threaded spindles. In the embodiment shown here, the support frame 15 is connected via four lifting columns 14 of the lifting system 8 to the holding frame 7, which is fastened to the floating body 3. The photobioreactor container 2 mounted on the support frame 15 can be lowered into the surface water 200 and also raised again by means of the lifting system 8. The support frame 15 can be moved vertically up and down within the recess 5 by rotating the threaded spindles. In addition, a collecting container 12, a pump unit 10 and a measurement and control unit 9 are arranged on the floating body 3. The collecting container 12 is used, for example, to harvest the microalgae culture used in the photobioreactor container 2. A pipeline 19 on the floating body 3 leading to the edge of the recess 5 connects the pump unit 10 to the lower outlet of the collecting container 12. A connection 41 for electrodes and a connection 42 for sampling are provided in said pipeline 19. A further pipeline 20 leading to the edge of the recess 5 arises from an upper outlet of the collecting container 12. The ends 23, 24 of the pipelines 19, 20 towards the recess 5 can be connected to the input 21 and output 22 of the pipe or pipe system 6 of the photobioreactor container 2. Not depicted here are a connection in the region of the input 21 of the pipe or pipe system 6 of the photobioreactor container for the supply of a gas or gas mixture, for example, an air/CO₂ mixture through a pneumatic hose, which is also not depicted, and a connection in the region of the output 22 of the pipe or pipe system 6 of the photobioreactor container 2, via which the gas or liquid can be removed from the photobioreactor container 2. The connections, which are preferably made by means of flexible lines, for example, pneumatic hoses, suction or pressure hoses and/or spiral hoses, so that the photobioreactor container 2 can be moved up and down without the fluid communication between the pipelines 19, 20 located on the floating body and the photobioreactor 2 being interrupted or hindered, are not depicted here. If the input 21 and the output 22 of the pipe or pipe system 6 of the photobioreactor 2 are connected by means of flexible connecting pieces to the ends 23, 24 of the pipelines 19, 20 located at the edge of the recess 5, a closed system that includes the photobioreactor 2 and the collecting container 12 is implemented. Both the collecting container 12 and the pipelines 19, 20 can be made of a transparent material, for example, plastic material.

This is not necessary, however. The culture medium can be circulated in the closed system by means of the pump unit 10. The measurement and control unit 9 is arranged on a frame 13 which is arranged on the floating body 3 and which is preferably a metal frame. The measurement and control unit 9 serves, among other things, for mixing the air/CO₂ mixture using appropriate flow meters, for power distribution, for optional manual control, for controlling the pump unit 10, for processing sensor data and for controlling the lifting system 8. Measurement data recorded by the measurement and control unit can also be transmitted wirelessly, for example, by radio (for example, WLAN), to a control system arranged outside the photobioreactor arrangement 1 and which comprises a control program with which, for example, remote control of the photobioreactor arrangement 1 and/or reading a measurement is made possible. In addition, a compressor 40 is arranged on the floating body 3, with the aid of which compressor 40 the microalgae culture can be aerated, for example, with an air / carbon dioxide mixture. The outer edge of the floating body 3 is surrounded by a railing 31 with the exception of a region 33 which is kept free and which serves to facilitate access to the photobioreactor arrangement 1. Two floating body modules 32, which act as jetties, are arranged here. A water passage 34 is arranged in the floating body 3, which water passage 34 is implemented here by a recess arranged in the lower part of the floating body 3 directed towards the water surface. A corresponding recess on the opposite side of the floating body 3 cannot be seen here. The recess can be implemented, for example, by inserting a floating body module 32 having a smaller extension in the vertical direction, so that when the floating body module 32 is connected to the remaining floating body, the floating body module 32, with its upper surface, is aligned with the upper surface of the remaining floating body 3 but does not, with its lower surface, contact the surface of the surface water 200. The water passage 34 serves for the exchange of water between the water within the recess 5 and the water of the surface water 200 which surrounds the floating body 3.

A microalgae culture can be cultivated in the photobioreactor container 2 when there is high solar radiation and high air temperatures. If necessary, the culture can be circulated by means of the pump unit 10. In order to produce optimal growth conditions with regard to light irradiation and temperature, the photobioreactor container 2 can be lowered into or raised from the surface water 200 by means of the lifting system 8. It is also possible to supply the culture with carbon dioxide or with air that is enriched with carbon dioxide. Connections to the pipe or pipe system 6 of the photobioreactor container 2 suitable for this purpose are here, as mentioned, not depicted in detail.

The culture used can then be harvested depending on the type of microalgae and other conditions, for example, after 7 to 21 days. The harvested biomass can then be further processed in a suitable manner, depending on the desired product, for example, washed and dried or digested.

FIG. 2 shows a top view of the embodiment depicted in FIG. 1 of a photobioreactor arrangement 1 according to the invention. To avoid unnecessary repetition, reference is therefore made to the description of FIG. 1 at this point.

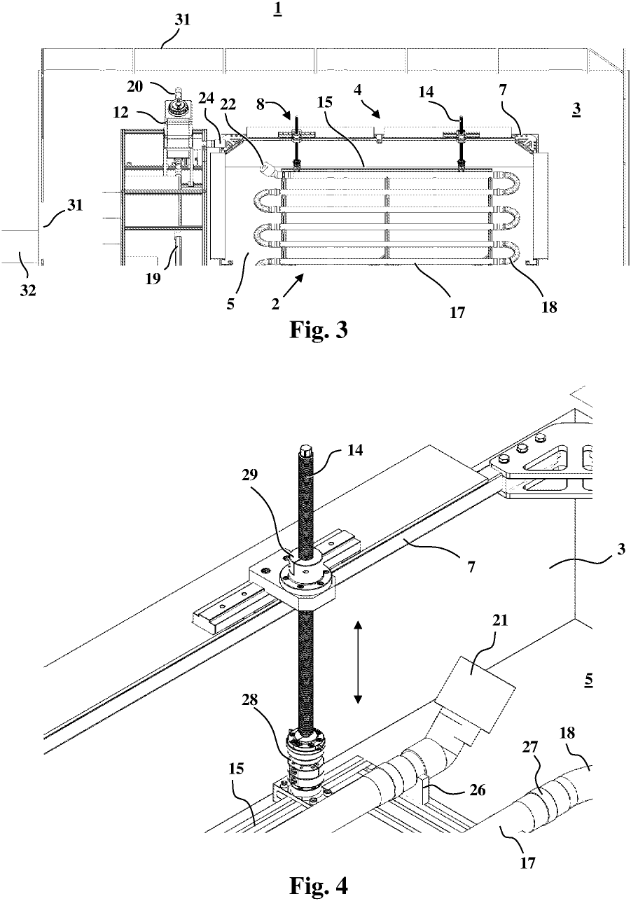

FIG. 3 shows a three-dimensional view of part of the embodiment of a photobioreactor arrangement 1 according to the invention depicted in FIGS. 1 and 2. The section shows, from a different perspective than that in FIG. 1, the region of the floating body 3 with the collecting container 12 and the pipelines 19, 20 leading therefrom to the recess 5, only the end 24 of the pipeline 20 arising from the upper part of the collecting container 12 being able to be seen here. A flexible line as a connection between the end 24 of the pipeline 20 and the output 22 of the pipe or pipe system 6 of the photobioreactor container 2 is also not depicted here. Two of the four lifting columns 14 of the lifting system 8 and part of the support frame 15 on which the pipe or pipe system 6 of the photobioreactor container 2 is mounted can be seen.

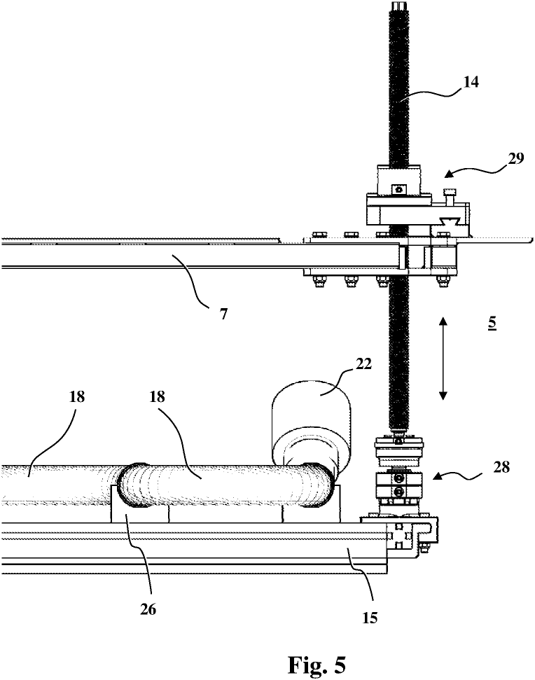

FIGS. 4 and 5 show a detailed view of part of the lifting system 8 having the lifting columns 14 comprising threaded spindles here. FIG. 4 shows a three-dimensional view of a lifting column 14 in the region of the input 21 of the pipe or pipe system 6 of the photobioreactor container 2, FIG. 5 shows a side view of a lifting column 14 in the region of the output 22 of the pipe or pipe system 6 of the photobioreactor container 2. Rotating the threaded spindles of the lifting columns 14 can move the support frame 15 in the vertical direction, that is, lowered or raised, as indicated by the arrow in the two figures. The threaded spindles can be rotated manually or by means of a motor, for example, an electric motor. The pipe or pipe system 6 of the photobioreactor container 2 is mounted on the support frame 15. For this purpose, the support frame 15 here has supports 26 having a recess which is circular in cross-section and which can accommodate cylindrical pipe sections of the pipe or pipe system 6. The lifting columns 14 are each fastened to the support frame 15 and to the holding frame 7 with fastening means 28, 29 in a suitable manner. The fastening means 29 with which the lifting columns 14 are fastened to the holding frame 7 comprise a ball-and-socket joint in order to avoid stresses during lifting and lowering and to enable the support frame 15 to incline and thus allow inclined lowering.

1. A photobioreactor arrangement (1), the photobioreactor arrangement (1) being buoyant on a surface water (200), comprising
a) a transparent photobioreactor container (2),
b) a floating body (3) that is buoyant on the surface water (200) and
c) a holding device (4) arranged at or on the floating body (3) holding the transparent photobioreactor container (2),
the photobioreactor container (2) being able to be lowered into the surface water (200) by means of the holding device (4).

2. The photobioreactor arrangement (1) according to claim 1, wherein i) the floating body (3) is arranged like a frame around the photobioreactor container (2) and has a recess (5) accommodating the photobioreactor container (2), ii) the holding device (4) holding the photobioreactor container (2) is arranged around the recess (5) and iii) the photobioreactor container (2) is held in the recess (5) by the holding device (4) moveably in the vertical direction.

3. The photobioreactor arrangement (1) according to claim 1, wherein the photobioreactor container (2) comprises a horizontally arranged, closed pipe or pipe system (6) made of a preferably rigid, transparent wall material.

4. The photobioreactor arrangement (1) according to claim 3, wherein the pipe or pipe system (6) is arranged in a horizontally meandering shape.

5. The photobioreactor arrangement (1) according to claim 1, comprising a plurality of photobioreactor containers (2), wherein each of the photobioreactor containers (2) is held by an individual holding device (4) and each of the photobioreactor containers (2) are lowerable individually into the surface water (200) by means of the respective individual holding device (4).

6. The photobioreactor arrangement (1) according to claim 1, comprising a plurality of photobioreactor containers (2), wherein at least two of the photobioreactor containers (2) are held by a common holding device (4) and are lowerable together into the surface water (200) by means of the common holding device (4).

7. The photobioreactor arrangement (1) according to according to claim 1, wherein the holding device (4) comprises a holding frame (7) with a lifting system (8) to which the photobioreactor container (2) is fastened and moveable vertically within the holding frame (7).

8. The photobioreactor arrangement (1) according to according to claim 1, wherein the photobioreactor container (2) is adapted to being lowered manually into the surface water (200) by means of the holding device (4).

9. The photobioreactor arrangement (1) according to according to claim 1, wherein the floating body (3) is formed from a plurality of parts.

10. The photobioreactor arrangement (1) according to according to claim 1, wherein the photobioreactor arrangement (1) comprises a measurement and control unit (9) arranged on the floating body (3).

11. The photobioreactor arrangement (1) according to according to claim 1, wherein the photobioreactor container (2) has an input (21) and an output (22) which input (21) and output (22) being connected to a collecting container (12) arranged on the floating body (3) by means of flexible lines (11) and a pump unit (10) arranged on the floating body (3).

12. The photobioreactor arrangement (1) according to claim 11, wherein the pump unit (10) is connected to the input (21) and output (22) of the photobioreactor container (2) and the collecting container (12) such that a culture medium received in the photobioreactor container (2) can be circulated through the collecting container (12) by means of the pump unit (10).

13. The photobioreactor arrangement (1) according to claim 10, wherein the photobioreactor container (2) is adapted to being automatically lowered into the surface water (200) on the basis of measured values captured by means of the measurement and control unit (9).

14. A photobioreactor arrangement (1), comprising
a) a transparent photobioreactor container (2),
b) a buoyant body (3) and
c) a holding device (4) arranged at or on the body (3), the holding device (4) holding the transparent photobioreactor container (2) and including means to raise and lower the photobioreactor container (2),
wherein the buoyancy of the buoyant body (3) is sufficient to support the transparent photobioreactor container (2), when filled with culture medium, above the surface of water when the photobioreactor arrangement (1) is placed on water.

15. The photobioreactor arrangement (1) according to claim 14, further comprising
a measurement and control unit (9) arranged on the floating body (3), the measurement and control unit (9) connected to at least one sensor adapted to record a physical parameter of the culture medium and/or the environment, the measurement and control unit (9) connected to the holding device (4) to automatically signal the holding device (4) to raise or lower the photobioreactor container (2) on the basis of values supplied by the at least one sensor to the measurement and control unit (9).

16. The photobioreactor arrangement (1) according to claim 15, wherein said at least one sensor is at least one of temperature, flow, photo, pH, pO₂, pCO₂ sensors.

17. The photobioreactor arrangement (1) according to claim 14, wherein the buoyancy of the buoyant body (3) is buoyancy in salt water.

18. The photobioreactor arrangement (1) according to claim 14, wherein the buoyancy of the buoyant body (3) is buoyancy in fresh water.

19. The photobioreactor arrangement (1) according to claim 14, further comprising a collecting container (12) being arranged on the floating body (3) between a pump unit (10) and the photobioreactor container (2), which collecting container (12) is adapted to harvesting a microalgae culture.
